(19) United States
(12) Patent Application Publication
(10) Pub. No.: US 2005/0059096 A1
(43) Pub. Date: Mar. 17, 2005

(54) Methods for measuring peroxisome proliferation and peroxisomal induction

(75) Inventors: Ingrid M. Pruimboom-Brees, Ledyard, CT (US); Dominique J.J.E. Brees, Ledyard, CT (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/941,250

(22) Filed: Sep. 15, 2004

Related U.S. Application Data
(60) Provisional application No. 60/503,293, filed on Sep. 15, 2003.

(51) Int. Cl. G01N 33/53; G01N 33/567

(52) U.S. Cl. 435/7.2; 514/2

(57) ABSTRACT

This invention provides methods for detecting peroxisome proliferation and peroxisomal beta oxidation in animals and tissue samples.

### FIELD OF THE INVENTION

This invention relates to novel methods for the detecting peroxisome proliferation and peroxisomal beta oxidation in animals and tissue samples.

### BACKGROUND OF THE INVENTION

Peroxisomes are single membrane-bound intracellular organelles that are contained in virtually all eukaryotic cells. All peroxisomes contain H₂O₂-catabolizing catalases and are therefore involved in protecting cells from this toxic byproduct. Peroxisomes also contain flavin-containing oxidases. One such oxidase is acyl-CoA oxidase (AOX). AOX is the rate-limiting enzyme of the peroxisomal β-oxidation of medium, long, and very long chain fatty acids. In rodents, peroxisomes characteristically increase in number and volume when exposed to so-called peroxisome proliferators. Such an increase is particularly relevant for rodent liver cells (hepatocytes). The effect of peroxisome proliferators is mediated by a class of nuclear receptors known as Peroxisome Proliferator Activated Receptors (PPAR). PPARs become transcriptionally activated in the presence of peroxisome proliferators and fatty acids. Four different PPAR subtypes have been described: alpha (α), beta (β), gamma (γ) and delta (δ). A number of different types of compounds act as peroxisome proliferators including, inter alia, various drugs, pesticide, plasticizers, specific diets and other chemicals (Cattley et al., Regulatory Toxicology and Pharmacology, vol. 27, pp. 47-60) (1998). In rats and mice, these peroxisome proliferators induce a variety of adaptive changes in the liver. Two of these changes are of considerable interest in the mechanism of tumor formation: 1) oxidative injury from induction of peroxisomal enzymes; and 2) peroxisome proliferation-induced enhanced cell replication. AOX catalyzes the conversion of the acyl-CoA fatty acids, e.g., palmitoyl-CoA, into shorter fatty acids, e.g., 2-hexadecenoyl-CoA (the fatty acid product of palmitoyl-CoA metabolism), producing H₂O₂ in the process. AOX production can be increased by over one order of magnitude in rodent livers following treatment with peroxisome proliferators. Conversely, catalase production is never increased more than two-fold. This imbalance between H₂O₂ generation and degradation may lead to oxidative damage to the cell if any H₂O₂ escapes the peroxisome. There is other evidence of oxidative damage in peroxisome proliferator-treated liver cells (hepatocytes). Oxidative injury in peroxisome proliferator-treated rodents has been linked with the production of tumors. For example, production of H₂O₂ in AOX-transfected monkey kidney cells led to the transformation of those cells in vitro when they were treated with linoleic acid, an AOX substrate.

In addition to oxidative injury from peroxisome proliferation-stimulated oxidase induction, peroxisome proliferation-induced cell proliferation has also been implicated in rodent hepato-carcinogenesis.

There exists a need for new and useful methods of determining and analyzing peroxisome proliferation and peroxisomal enzyme activity are needed.

Historically, biochemical methods and electron microscopy are used to demonstrate peroxisome proliferation and peroxisomal induction. For example, cell extracts from tissue samples can be assayed for total protein content and AOX enzymatic activity. The results can be compared to control samples that contain non-proliferating, non-induced peroxisomes. However, traditional biochemical methods have the following disadvantages: 1) they do not enable true determination of peroxisome proliferation, but only induction; 2) they are time consuming; 3) because tissue samples must be homogenized in order to obtain cell extracts for analysis, the samples are destroyed and tissue architecture is lost in the process, and re-examination of the same tissue is impossible; 3) the localization of specific tissue regions in which peroxisome induction has occurred cannot be determined; and 4) they often involve the use of hazardous substances, e.g., radioactive labels.

Electron microscopy (EM) is often used to confirm the results of the biochemical experiments. While this technique has the advantage (over biochemical methods) of preserving the architecture of the tissue samples, electron microscopy has the following disadvantages: 1) EM is time consuming because it requires special fixatives and techniques for processing the sample and preparing the slide for microscopy; 2) only a limited number of cells can be examined using electron microscopy; 3) complex, time consuming image analyses are needed in order to process the data and electron micrographs; 4) only peroxisome proliferation, and not peroxisomal induction, can be analyzed using electron microscopy; and 5) samples are consumed during the process of generating electron micrographs and thus cannot be archived and re-used later.

Methods that can be used to determine and analyze peroxisome proliferation and peroxisomal enzyme activity without the above-mentioned disadvantages of traditional biochemical methods and electron microscopy are desirable.

### SUMMARY OF THE INVENTION

This invention provides novel methods to measure peroxisome proliferation and the induction of peroxisomal enzyme activity. The methods according to this invention overcome the above-mentioned deficiencies encountered when employing traditional biochemical methods and electron microscopy.

The novel methods according to this invention use a technique called Laser Scanning Cytometry (LSC) to measure peroxisome proliferation and/or peroxisomal induction using immunohistochemical detection of two peroxisome markers: 1) a conserved peroxisome protein tri-peptide marker called the peroxisomal targeting signal (PTS-1); and 2) AOX.

### BRIEF DESCRIPTION OF THE DRAWINGS

FIG. 1 is a photomicrograph showing liver tissue from control rats (untreated with peroxisome proliferators) that are stained with a green fluorescent dye (FTIC) that has been conjugated to an antibody specific for AOX. The sparse fluorescence is due to the low number of peroxisomes and AOX activity in the hepatocytes of the control rats. Cell nucleuses are stained with DAPI and appear blue.

FIG. 2 is a photomicrograph showing liver tissue from a rat treated with a peroxisome proliferator. The tissue has been stained using a green fluorescent dye (FTIC) that has been conjugated to an antibody specific for AOX. The strong fluorescence relative to that seen in FIG. 1 is due to the increased number and volume of the peroxisomes in the hepatocytes (peroxisome proliferation), as well as increased AOX activity (increased intensity of staining due to peroxisomal enzyme induction).

FIG. 3 is a photomicrograph showing liver tissue from a rat treated with a peroxisome proliferator. The tissue has been stained using a green fluorescent dye (FTIC) that has been conjugated to an antibody specific for PTS-1, a tri-peptide that is conserved in all peroxisome proteins, including AOX.

FIG. 4 are two LSC scattergrams (XY plits, and MPI versus integral plot) depicting PTS-1 activity in control rats that have not been treated with a peroxisome proliferator. Top Panel, XY plot: Hepatocytes displaying PTS1 immunofluorescence were mapped to visualize tissue architecture and relocated to confirm that data scattergrams were correctly interpreted. Bottom Panel: FITC-MPI versus FITC-Integral Analysis data was plotted for each scan. The graph shows that 0.4% (0.3+0.1) of hepatocytes have high levels of PTS-1 activity (arrows).

FIG. 5 are two LSC scattergrams (XY plits, and MPI versus integral plot) depicting PTS-1 activity in rats that have been treated with a peroxisome proliferator. Top XY plot: Each hepatocyte is given X and Y coordinates. Relocation is performed to confirm the level of fluorescence and to confirm that cells are truly immunofluorescent. Bottom plot: MPI versus Integral analysis is displayed for each scan. The graph shows that 15.5% of hepatocytes (13.7+1.7+0.1) have increased PTS-1 activity, especially in the centrilobular areas.

FIG. 6 is a photomicrograph showing the peroxisomal enzyme (AOX) immunohistochemistry (FITC) in the livers of the control dogs, i.e, dogs that have not been treated with a peroxisome proliferator.

FIG. 7 is a photomicrograph showing the peroxisomal enzyme (AOX) immunohistochemistry (FITC) in the liver of dogs that have been trated with a peroxisome proliferator. The intensity/brightness of the FITC fluorescence is higher in treated dogs (FIG. 7) than in control dogs (FIG. 6).

### DETAILED DESCRIPTION OF THE INVENTION

In order to more fully understand the invention herein described, the following detailed description is set forth. In the description, the following terms are employed:

Flow cytometry (FC)—a method of measuring fluorescence from stained cells that are in suspension and flowing through a narrow tube. FC uses one or two lasers to activate (excitation and emission). The cellular target of interest is labeled using a fluorescence tag (either via immunochemical or other means), which becomes excited treated with a laser. Upon return to a relaxed state the excited fluorescence molecule releases an emission wavelength which is detected by the instrument and translated to an electronic signal by and measured. The more fluorescence the higher the intensity of the signal and the higher the reading is. FC can be used to determine multiple cellular constituents and other features of cells, but cannot be used to determine features of tissue samples.

Fluorescein isothiocyanate (FITC) and 4′,6-diamidino-2-phenylindole (DAP1)—FITC and DAPI are fluorochromes that can be used as fluorescent tags or markers. Fluorochromes are chemicals that emit light at one wavelength (called emission) when exposed to light at another wavelength (called incident light). For example, when a laser is applied to cells that contain an FITC-conjugated (tagged) antibody, the FITC becomes excited. As it relaxes, the FITC emits green fluorescent light. DAPI is a dye that acts as a nuclear marker because it is able to get into a cell's nucleus and bind DNA. When properly excited, DAPI emits blue fluorescent light. Fluorochromes such as FITC and DAPI are used as fluorescent tags in biochemistry and immunology.

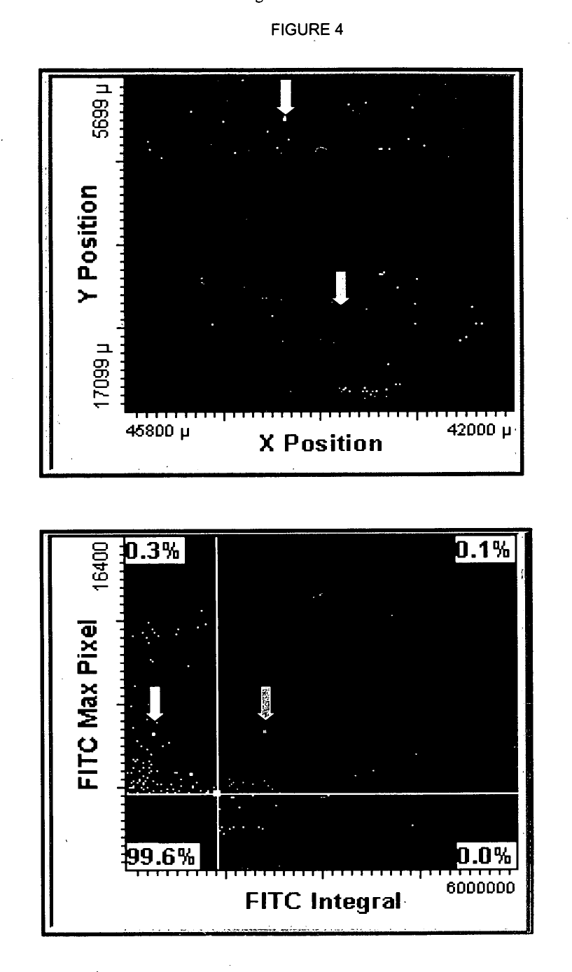

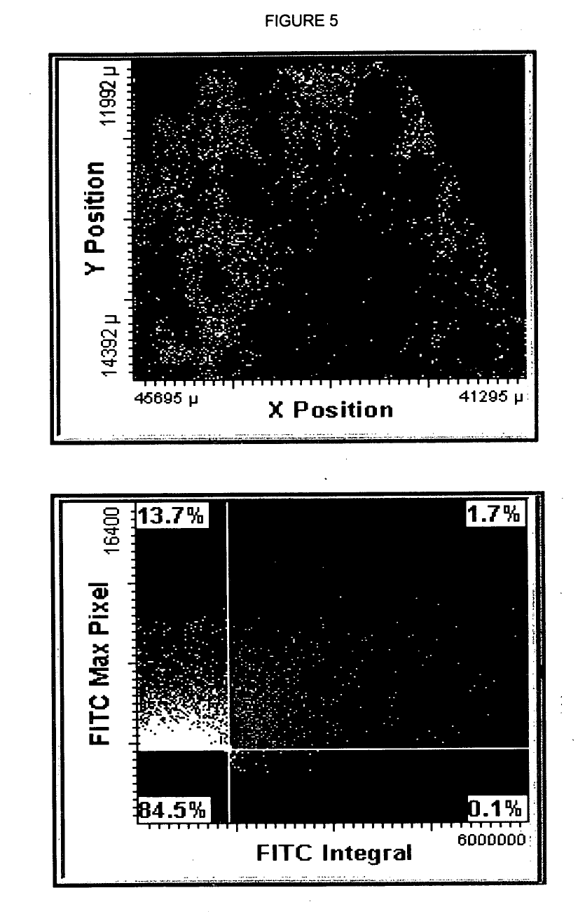

Gating—gating is a method of defining whether or not a specific fluorescence reading is above or below background fluorescence, or represents baseline fluorescence from a normal cell (one without peroxisome proliferation or peroxisomal induction), or represents fluorescence above background and baseline (i.e., a cell wherein the peroxisomes have been stimulated to proliferate or wherein peroxisomal enzymes have been induced). For the examples herein, cells that have little to no peroxisome staining appear at the bottom (lower left hand corner) of the graph because they exhibit little green fluorescence. Because these cells are resting or normal cells (no proliferation or peroxisomal induction), gates (a vertical and horizontal bar which ends up making four quadrants) may be applied around the areas containing them. The gates basically indicate what the limit of fluorescence is in a normal or resting cell. Once gates are applied to these areas, the level of background and/or baseline fluorescence has been determined, and these levels will be applied to data obtained in the study. Thereafter, any fluorescence readings with values above this background or baseline levels will be representative of peroxisome proliferation or peroxisomal induction. These cells will be evident because they will appear in one of the quadrants other than the “gated” lower left-hand quadrant of the graph (usually, but not always, in the upper left-hand and upper right-hand quadrants). The data is processed in such a way that each quadrant of the plot will display the percentage of total cells in that quadrant. For example, see FIGS. 4 and 5, right panels. In the right-side panel of FIG. 4 (which represents PTS-1 activity in control rats), it is apparent that background is 99.6% (lower left quadrant) and that 0.4% (0.1+0.3) of hepatocytes appear in the upper left and right quadrant. This is consistent with baseline expression of PTS1 in control hepatocytes. Conversely, in the right-side panel in FIG. 5, representing PTS-1 activity in rats that have been treated with peroxisome proliferators), 15.4% (13.7+1.7) of the cells examined appear in the upper left-hand and upper right-hand quadrants, with very high MPI and integral values (compared to FIG. 4). This is consistent with peroxisomal induction and proliferation.

Immunohistochemistry—This method uses antibodies against specific cellular components to detect their localization in tissue. The antibodies are incubated on the tissue sections for the time necessary to recognize the antigen (cellular target). Using laser scanning cytometry, the antibody can be detected directly if it is conjugated to a fluorescence tag. The antibody can also be detected or indirectly using a chemical reaction causing the colorimetric reaction of a chromogen.

Integral Analysis—instead of measuring the brightness of every fluorescent dot, as is done to determine MPI (see definition of MPI, below), the apparatus instead measures the total fluorescence per cell (sum of all the fluorescent dots within each cell). Integral analysis (X axis) is plotted against MPI (Y axis) in order to obtain the plots seen in the right-side panels of FIGS. 4 and 5.

Laser Scanning Cytometry (LSC)—LSC is a microscope-based cytometry method that automatically measures laser-excited fluorescence at multiple wavelengths and light scatter from cells in order to rapidly (within minutes) determine multiple cellular constituents and other features, e.g., enzymatic activity, protein presence, and number and size of organelles. Because the measurements are microscope slide-based, measures cells on the surface of the slide, and obtains higher resolution than other cytometry methods, LSC has certain advantages over other techniques, e.g., flow cytometry (FC; see above), immunohistochemistry (see above) and traditional biochemical methods. Furthermore, LSC has certain attributes that are very relevant to the methods according to this invention: (1) LSC is capable of examining tissue samples without disrupting the native tissue architecture; (2) LSC is capable of simultaneously examining many thousands of cells in a sample, (3) LSC can be used to determine the location within tissue where peroxisomal activation is occurring in the context of the native architecture of the tissue where they are located and compare then to other tissue features including, inter alia, blood vessels and Kupffer cells; and (3) LSC can differentiate between peroxisomal induction and proliferation.

Maximum Pixel Intensity (MPI)—MPI represents the maximal fluorescence intensity, or brightness. To obtain this variable, the apparatus being used to measure fluorescence determines the intensity of blue or green fluorescence for every one of the fluorescent dots in the data set. Herein, the blue MPI reading (from DAPI) represents each cell nucleus in the tissue section being examined, while the green MPI reading (from FITC) represents each peroxisome in the tissue section being examined. MPI is plotted (Y axis) against the values obtained for integral analyses (X axis; see definition of integral analysis, above) in order to obtain the plots seen in the right-side panels of FIGS. 4 and 5.

Peroxisomal Induction—the induction of peroxisomal enzymes including, inter alia, AOX, in response to peroxisome proliferators. Peroxisomal induction can be measured by the methods according to this invention.

Peroxisome Proliferation—an increase in the number and the size of the peroxisomes within a cell, in a response to peroxisome proliferators. Peroxisome proliferation can be measured by the methods according to this invention.

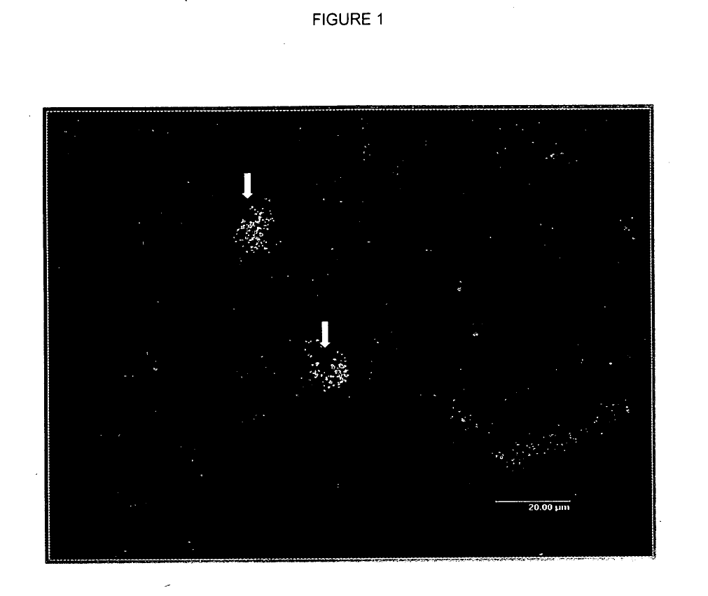

Relocation—relocation refers to a technique used to confirm data points of high intensity fluorescence. This technique allows the investigator to make sure that a data point of high fluorescence is due to peroxisome proliferation and/or induction and not due to an experimental artifact, e.g., incomplete washing of the unbound labeled antibody off of the sample. For example, see FIG. 4. The left-hand panel shows a spatial distribution of hepatocytes in the two liver samples being examined, wherein each hepatocyte has been assigned a pair of XY coordinates. The right-hand panel shows a plot of MPI (see definition, above) versus total fluorescence (integral analysis; see definition, above). In the right-hand panel, two hepatocytes appear that have, respectively, an MPI (hepatocyte marked with a white arrow) or a total fluorescence (hepatocyte marked with a green arrow) that are above background (i.e., outside the gated lower left-hand quadrant; see definition of gating, above). Relocation allows for the determination of whether these “above-background” data points are due to peroxisomal proliferation and induction, or are merely artifacts: because each hepatocyte has been assigned coordinates and can therefore be relocated in the spatial distribution of the tissue sample in the left-hand panel, the investigator can ask a computer to take the hepatocytes in the right-hand panel and relocate them in the left-hand panel. This has been done and the same hepatocytes have been marked, respectively, with a white and green arrow in the left-hand panel of FIG. 4. Now, a photograph of the individual hepatocyte of interest can be taken and examined to determine the validity of the data. This has been done for the hepatocyte marked with the green arrow in FIG. 4. A photograph of this same hepatocyte appears in FIG. 1, which shows that the high-fluorescence signal is not an artifact but is instead due to peroxisomal induction (seen as two clusters of fluorescent peroxisomes marked with green arrows in FIG. 1).

Scattergram—a graph with points plotted on a coordinate plane. A scattergram according to this invention is a graph that locates individual cells, e.g., hepatocytes, in tissue slides used in LSC, by assigning each cell X and Y-coordinates.

XY Plots—XY plots, such as those seen in the left-hand panels of FIGS. 4 and 5, display the spatial distribution of every cell, e.g., hepatocyte, observed in the tissue sample. The X and Y axes represent the length and width, respectively, of the microscopic slide. Two sections (one roughly square and the other roughly trapezoidal; see FIGS. 4 and 5, left-hand panels) of tissue, e.g., liver tissue, are examined in a typical experiment. Every cell, e.g., hepatocyte, in each section of the sample liver is given an XY coordinate. All the hepatocytes are then “plotted” on the graph and that give you the 2 microscopic sections of the liver.

Protein transport into cellular organelles requires the translocation of proteins across one or more biological membranes. In peroxisomes, protein transport is mediated by cis-acting targeting signals present within the imported proteins. The carboxy-terminal of peroxisome proteins is composed of a highly conserved tripeptide with the sequence serine-lysine-leucine (Ser-Lys-Leu) that is referred to as the peroxisomal targeting signal (PTS-1) (Gould et al., The Journal of Cell Biology, vol. 110, pp. 27-34 (January 1990)). A protein containing the PTS-1 is transported into a peroxisome by binding to a PTS-1 receptor or to one or more so-called docking proteins of the peroxisome translocation apparatus (Rachubinski and Subramani, Cell, vol. 83, pp. 525-28 (1995)). In contrast to proteins transported into the endoplasmic reticulum and mitochondria, covalent modification of peroxisome proteins does not occur as a consequence of importation into the peroxisome matrix. Therefore, the Ser-Lys-Leu sequence remains in the protein after it is transported to the peroxisome. Consequently antibodies may be raised against this highly conserved PTS-1 sequence. This allows the PTS-1 sequence to be used in immunohistochemistry experiments (Usuda et al., The Journal of Histochemistry & Cytochemistry, vol. 47(9), pp. 1119-26 (1999)). Antibodies against AOX may also be raised, allowing AOX to be used in similar experiments. Therefore, immunohistochemical detection of PTS-1 or AOX may be used to localize peroxisomes within cells.

Laser scanning cytometry (LSC) is a new laboratory technique that combines properties and advantages of flow cytometry (FC) and immunohistochemistry (IHC) and provides qualitative and quantitative information on DNA content, protein expression and cellular localization. Immunohistochemistry uses antibodies directed against specific cellular components in order to detect their localization in tissue samples. After incubating the antibodies with the tissue samples, the bound antibody can be detected directly if it has been tagged with a fluorescent marker. This is possible because LSC is a microscope slide-based method, and the procedure used to prepare the tissue samples for LSC allows for the antibodies to access their antigens even though the antigens are located within membrane-bound organelle. More specifically, the tissue samples are fixed in a special fixative called formalin. Doing this prevents the tissue and its components from being degraded. The tissue is then embedded in paraffin to harden it for cutting. 5 μm (thickness) thin-layer sections of the paraffin-embedded tissue samples are then cut and placed on the microscope slides. The antibody of interest is then added directly to the slide and the slide is incubated. Because the tissue samples are cut in to 5 μm-thick slices, while a normal cell is about 20 μm thick, the cytoplasm and organelles are “laid open” and the antibody can get inside the organelles. This is all accomplished without disrupting the architecture of the tissue, because the tissue was fixed in formalin. If needed, a detergent that will increase membrane permeability without disrupting the architecture of the tissue may be added.

Both LSC and FC can be used to measure fluorescence signals in cells or in organelles within cells, e.g., peroxisomes. For instance, a liver tissue sample can be treated with an antibody specific for a peroxisome protein such as AOX. If the antibody has been conjugated with a fluorescent tag, e.g., fluorescein isothiocyanate (FITC), any cells containing AOX can be visualized after exposure to a laser under a fluorescence microscope. Two types of analysis can be performed in this manner: 1) peroxisome proliferation can be determined by counting the number of stained peroxisomes under the microscope, and measuring the intensisty (MPI) and the total fluorescence (integral) of the peroxisome; and 2) induction of peroxisomal enzyme, e.g., AOX, activity can be determined by measuring the intensity of the peroxisomal staining. LSC has some advantages over FC. FC measures cells in suspension as they flow past a laser source and into a waste container, while LSC measures cells that are maintained in their positions within the overall architecture of the tissue sample. Therefore, samples for use in LSC can be archived and re-used and cells may be measured in their native tissue environments, i.e., in situ.

In a preferred embodiment, the methods according to this invention can be used to determine whether a test compound is capable of causing peroxisome proliferation and/or peroxisomal induction.

In another embodiment, the methods according to this invention can be used to determine whether a test organism has been exposed to a compound capable of causing peroxisome proliferation and/or peroxisomal induction.

In yet another embodiment, the methods according to this invention can be used to detect whether peroxisome proliferation and/or peroxisomal induction has occurred in a test organism.

In yet another embodiment, the methods according to this invention can be used to detect or diagnose diseases or disorders in organisms including, inter alia, humans, that are the consequence of peroxisome proliferation and/or induction. Examples of such diseases and disorders include, but are not limited to, tumorigenesis and cancer, oxidative injuries, liver disease, heart disease and kidney disease.

### EXAMPLES

In order that this invention may be better understood, the following examples are set forth. These examples are for the purpose of illustration only and are not to be construed as limiting the scope of the invention in any manner.

### Example 1

### Analysis of Peroxisome Proliferation and Induction of Peroxisomal Enzymes in Rat Liver

Anti-AOX and PTS-1 Antibodies:

For the anti-PTS-1 antibody, a PTS-1 peptide with the sequence CRYHLKPLQSKL (one-letter amino acid designation) was synthesized and conjugated to keyhole limpet hemocyanine (KLH), as previously described (see Gould et al., cited above). Rabbit polyclonal antibodies were raised against the conjugated PTS-1 peptide and the serum with the highest titers was used for all subsequent immunofluorescence experiments.

The antibody specific for AOX was a generous gift from Dr. Nobuteru Usuda and was prepared by immunizing rabbits with AOX purified from rat liver tissues (Usuda et al., The Journal of Histochemistry & Cytochemistry, vol. 47(9), pp. 1119-26 (September 1999)).

Animals and Tissue Preparation:

Sprague-Dawley rats were treated with a potent peroxisome proliferator by oral gavage daily for 30 consecutive days. The rats were divided into three test groups, which were given 1, 3, and 10 milligrams (mg), respectively, of the peroxisome proliferator, per kilogram (kg) of body weight. After the 30 day period, the rats were fasted overnight, anesthetized with CO₂, and killed. Following necropsy, the right and left liver lobes were collected and divided into three fractions. Fraction 1 of the collected liver tissue samples was immediately frozen for later use in biochemical experiments. Fraction 2 was fixed in 10% formalin buffer, embedded in paraffin blocks and processed for hematoxylin and eosin (H&E) staining and examined by light microscopy. Fraction 3 was fixed in 10% formalin buffer, and embedded in paraffin blocks for later use in laser scanning cytometry experiments.

Immunofluorescence:

5-micron (μm)-thick liver sections were cut from the paraffin blocks, de-paraffinized using xylene rehydrated in alcohol, and washed in OptiMax wash buffer (OWB) (Biogenex, San Ramon, Calif.). The liver sections were then placed on slides. Proteolytic digestion was performed by incubating the slides for 12 minutes at 37° C. in Pepsin (Dako Corporation, Carpeinteria, Calif.). Slides were rinsed three times for three minutes in OWB.

Nonspecific binding sites were blocked by the addition of 5% normal donkey (Jackson Immuno Research Laboratories, Inc.) serum in OWB for 20 minutes at room temperature (approximately 20° C.). Sections were then incubated for 1 hour in a humid chamber with either of the following antibodies: (1) rabbit anti-AOX (diluted 1:3000); or (2) rabbit anti-PTS-1 (diluted 1:500). Sections were washed in OWB three times for three minutes. Secondary donkey anti-rabbit IgG fluorescein isothiocyanate (FITC) conjugated antibody was added for 30 minutes and slides were placed in a humid chamber at 37° C. Slides were washed three times for three minutes in OWB, followed by coverslipping in Vectashield mounting medium with 4′,6-diamidino-2-phenylindole (DAPI) (Vector laboratories, Burlingame, Calif.). Negative control slides were obtained by omitting the incubation with the primary antibody, followed by treatment with FITC alone (in order to measure background FITC fluorescence).

Analysis of Cell Fluorescence by LSC:

Slides were inspected under an epifluorescence microscope (Olympus BX50) using a mercury lamp illumination. The fluorescence of at least 8000 cardiomyocytes and 20,000 hepatocytes was measured by LSC (the LSC apparatus from CompuCyte Corp., Cambridge, Mass.) at 20× magnification. Briefly, FITC and DNA-associated DAPI fluorescence were excited with an argon ion laser (at a wavelength of 488 nanometers (nm)) operating at 5 milliwatts (mW) and a violet diode laser (at a wavelength of 400 nm). Green (FITC) and blue (DAPI) fluorescence were collected by separate photomultipliers. Individual hepatocytes were identified with nuclear DAPI fluorescence (identified by a fluorescent color), and cytoplasmic borders were selected at 10 pixels (corresponding to an area of approximately 5 μm at 20× magnification) outside the nuclear contour on the basis of the FITC fluorescence. For each cellular contour, the values of green and blue fluorescence intensities were measured and automatically processed to generate integral analysis, maximum pixel intensity (MPI), and XY coordinates of individual hepatocytes. Negative control slides were used to determine the level of background fluorescence while control rats were used to define gating prior to analysis (gating means that you define when a fluorescent event is considered either above background fluorescence or baseline expression in a control cell—in the case of PTS-1 and AOX fluorescence, gating was done to separate background fluorescence from expression in control or treated hepatocytes or cardiomyocytes). AOX- or PTS-1-expressing hepatocytes were mapped to visualize tissue architecture (X-Y plot) and relocated to confirm that the data on the scattergrams (a graph with points plotted on a coordinate plane) were correctly interpreted. A scattergram that plotted FITC-MPI (Y position) versus FITC-integral analysis (X position) was displayed for each scan. The results from the LSC analysis were compared to the biochemical assay that is the traditional method used for peroxisomal β oxidation measurements.

Results:

Immunofluorescence of Rat Hepatocyes as Observed by Microscope Attached to a Laser Scanning Cytometer:

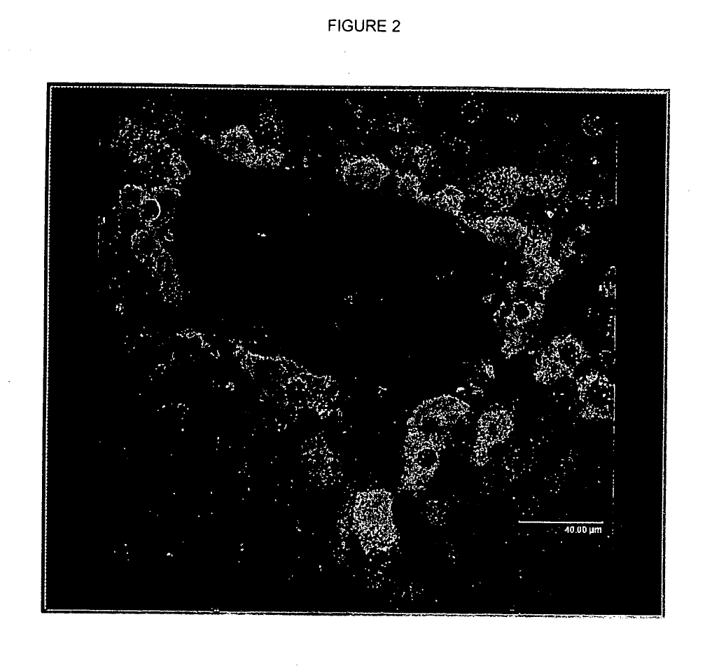

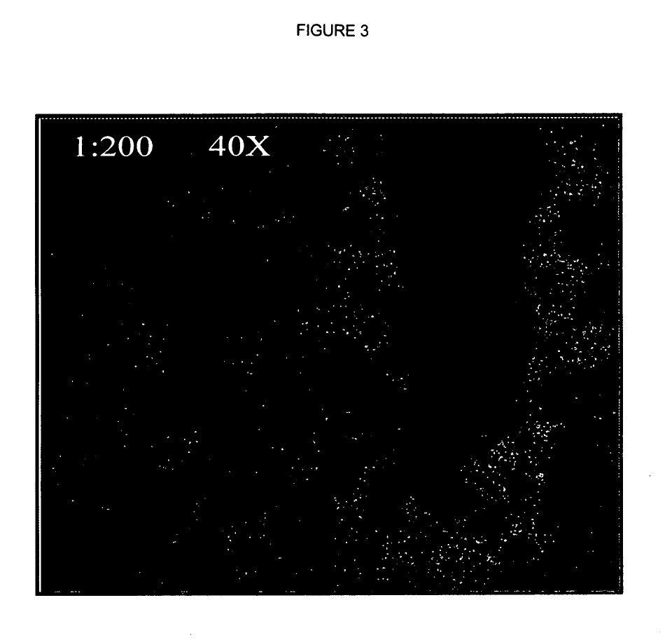

In control rats, occasional centrilobular or midzonal hepatocytes of normal size showed intense PTS-1 and AOX immunofluorescence that was associated with low number of cytoplasmic organelles of a size (roughly 0.5-1 μm in diameter) consistent with peroxisomes (FIG. 1). In peroxisome proliferator-treated rats (see FIG. 2 (AOX activity) and FIG. 3 (PTS-1)), there was a strong increase in immunofluorescence in the centrilobular and midzonal hepatocytes due to increases in peroxisome number and volume (i.e., peroxisome proliferation, seen as increased number and volume of the green dots in FIGS. 2 and 3), and FIG. 2 also shows an increase in peroxisomal enzyme (AOX) activity (i.e., peroxisomal induction, seen as an increase in intensity or brightness (increased MPI) of the green dot fluorescence). FIG. 3 also shows a strong increase in the presence of PTS-1 (i.e., peroxisomal protein induction, seen as an increase in intensity or brightness (increased MPI) of the green dot fluorescence).

Analysis of Rat Hepatocyte Fluorescence by LSC:

For each cellular contour, the values of green and blue fluorescence intensities were measured and automatically processed to generate integral analysis, MPI, and XY coordinates of individual hepatocytes. Negative control slides were used to determine the level of background fluorescence while data from control rats (those untreated with peroxisome proliferator) were used to define gating prior to analysis. PTS-1-expressing rat hepatocytes from control rats (FIG. 4, left panel) and peroxisome proliferator-treated rats (FIG. 5, left panel) were mapped to visualize tissue architecture and relocated to confirm that data on the scattergrams were correctly interpreted. A scattergram plotting FITC-MPI data versus FITC-integral data was generated for each scan (FIGS. 4 and 5, right panels). The results demonstrate that PTS-1 activity is much greater in rats that have been treated with a peroxisome proliferator (FIG. 5) than in control rats that have not been treated with a peroxisome proliferator (FIG. 4).

Comparison with Traditional Biochemical Methods:

The results of the laser scanning cytometry experiment were compared to those obtained with a traditional biochemical method that measures palmitoyl-CoA (a substrate for AOX) beta-oxidation in the following manner:

Frozen sections from all liver samples were gradually thawed in Dulbecco's phosphate-buffered saline (D-PBS). Following the addition of cold 0.25M sucrose, samples were minced, homogenized, and centrifuged at 600×g for 10 minutes in a refrigerated rotor. Supernatants were frozen in an Ultralow freezer. On the day the assays were to be performed, supernatants were thawed and assayed separately for protein content and palmitoyl CoA activity. Total protein was determined by the bicinchoninic acid method. Following the addition of Triton X-100, each sample was mixed and then aliquots were transferred to a Cobas sample cup with reaction mixture and 0.5 mM palmitoyl CoA separately placed in the appropriate reagent cups. A 60 second incubation at 37° C. was followed by 30 absorbance readings at 340 nm taken at 10 second intervals in order to determine the change in a absorbance (“Δ Abs”) per minute. For each sample, the resulting Δ Abs/min was multiplied by a calculation factor F to yield μmol×L−1×min−1. This value was then multiplied by a homogenate dilution factor and divided by 1000 g/L to yield the final palmitoyl CoA activity as measured in μmoles of NAD reduced/g liver×minutes. This data was then compared to that obtained in the laser scanning cytometry studies. The results of the comparison are shown in Table 1:

TABLE 1
Summary data comparing the results from the biochemical assay and
laser scanning cytometry.

| | | | | PTS1 percentage | | AOX percentage | |
|---|---|---|---|---|---|---|---|
| | | □moles NAD | | “positive” | | “positive” | |
| | | reduced/g/min | | hepatocytes | | hepatocytes | |
| Treatment | | Males | Females | Males | Females | Males | Females |
| Control | Mean | 0.24 | 0.14 | 1 | 1.16 | 0.14 | 0.5 |
| | SD | 0.8 | 0.05 | — | — | — | — |
| 1 | Mean | 6.813 | 4.486 | 4.16 | 5.62 | 21.68 | 16.86 |
| mg/kg | SD | 0.72 | 0.48 | — | — | — | — |
| 3 | Mean | 5.834 | 4.186 | 3.5 | 5.48 | 30.8 | 28.52 |
| mg/kg | SD | 1.13 | 0.4 | — | — | — | — |
| 10 | Mean | 8.417 | 4.517 | 17.04 | 9.24 | 59.94 | 30.7 |
| mg/kg | SD | 1.31 | 0.34 | — | — | — | — |

As seen in Table 2, there is a very good correlation between the biochemical assay and either the PTS-1 or AOX results (see above). For example, At 1 and 3 mg/kg, the values of the biochemical assay (4.486 and 4.186 micromoles (μM), respectively, of NAD reduced/g/min) are almost the same in rats (there was not an increase in biochemical activity in rats at 1 and 3 mg/kg). Similarly, in the LSC study, the PTS-1 values are almost identical (5.62 and 5.48 of hepatocytes expressing a level of PTS-1 activity higher that in controls at 1 and 3 mg/kg, respectively) in rats given 1 and 3 mg/kg, respectively, of the peroxisome proliferator.

### Example 2

### Analysis of Peroxisome Proliferation and Induction of Peroxisomal Enzymes in Dog Liver

Anti-AOX and Anti-PTS-1 Antibodies:

Anti-AOX and anti-PTS-1 antibodies were prepared as in Example 1, above.

Animals and Tissue Preparation:

Three beagle dogs were treated with 50 mg/kg of body weight of a potent peroxisome proliferator by oral gavage daily for 30 consecutive days. Three dogs remained untreated for use as control animals. After the 30 day period, the dogs were fasted overnight, anesthetized with CO₂, and killed. Following necropsy, the right and left liver lobes were collected and fixed in 10% formalin buffer, processed for hematoxylin and eosin (H&E) staining and examined by light microscopy. Remaining liver tissues were frozen in paraffin blocks for later use.

Immunofluorescence:

5-micron (μm)-thick liver sections were cut from the paraffin blocks, de-paraffinized using xylene rehydrated in alcohol, and washed in OptiMax wash buffer (OWB) (Biogenex, San Ramon, Calif.). The liver sections were then placed on slides. Proteolytic digestion was performed by incubating the slides for 12 minutes at 37° C. in Pepsin (Dako Corporation, Carpeinteria, Calif.). Slides were rinsed three times for three minutes in OWB.

Nonspecific binding sites were blocked by the addition of 5% normal donkey (Jackson Immuno Research Laboratories, Inc.) serum in OWB for 20 minutes at room temperature (approximately 20° C.). Sections were then incubated for 1 hour in a humid chamber with either of the following antibodies: (1) rabbit anti-AOX (diluted 1:3000); or (2) rabbit anti-PTS-1 (diluted 1:500). Sections were washed in OWB three times for three minutes. Secondary donkey anti-rabbit IgG fluorescein isothiocyanate (FITC) conjugated antibody was added for 30 minutes and slides were placed in a humid chamber at 37° C. Slides were washed three times for three minutes in OWB, followed by coverslipping in Vectashield mounting medium with 4′,6-diamidino-2-phenylindole (DAPI) (Vector laboratories, Burlingame, Calif.). Negative control slides were obtained by omitting the incubation with the primary antibody, followed by treatment with FITC alone (in order to measure background FITC fluorescence).

Analysis of Cell Fluorescence by LSC:

Slides were inspected under an epifluorescence microscope (Olympus BX50) using a mercury lamp illumination. The fluorescence of from 8000 to greater than 20,000 hepatocytes per section of liver was measured (contour analysis) by LSC (the LSC apparatus from CompuCyte Corp., Cambridge, Mass.) at 20× magnification. Briefly, FITC and DNA-associated DAPI fluorescence were excited with an argon ion laser (at a wavelength of 488 nanometers (nm)) operating at 5 milliwatts (mW) and a violet diode laser (at a wavelength of 400 nm). Green (FITC) and blue (DAPI) fluorescence were collected by separate photomultipliers. Individual hepatocytes were identified with nuclear DAPI fluorescence (identified by a fluorescent blue color). For each cellular contour, the values of green (FITC) fluorescence intensities were measured and automatically processed to generate integral analysis, maximum pixel intensity (MPI), and XY coordinates of individual hepatocytes. Negative control slides were used to determine the level of background fluorescence while control dogs were used to define gating prior to analysis. A scattergram that plotted FITC-MPI (Y position) versus FITC-integral analysis (X position) was displayed for each scan (data not shown). The results from the LSC analysis were compared to the biochemical assay that is the traditional method used for peroxisomal β oxidation measurements.

Results:

Immunofluorescence of Dog Hepatocyes as Observed by Microscope Attached to a Laser Scanning Cytometer:

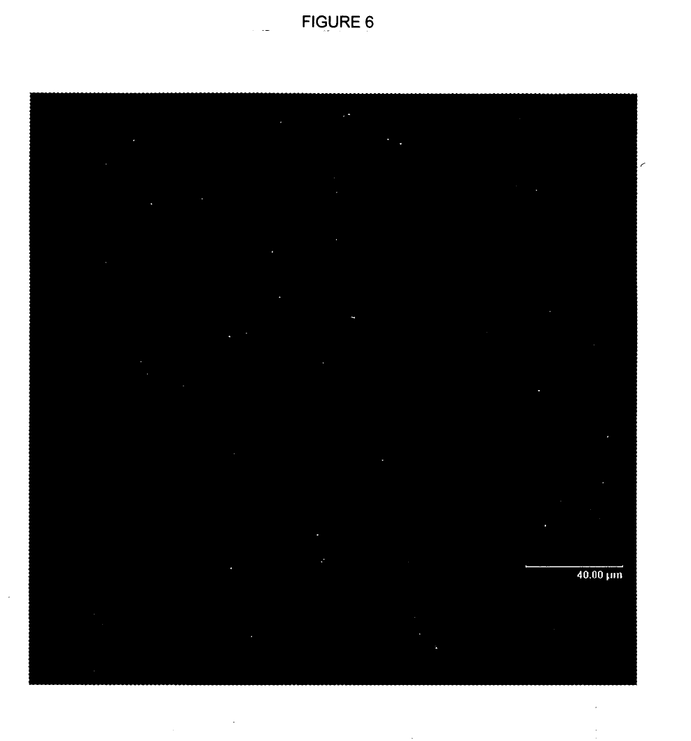

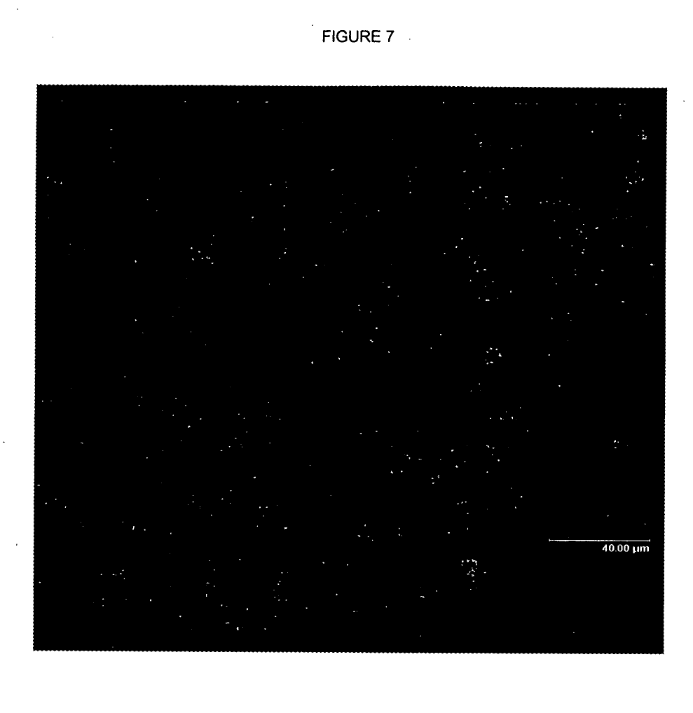

In peroxisome proliferator-treated dogs (FIG. 7), there was an increase in peroxisomal enzyme (AOX) activity (i.e., peroxisomal induction, seen as an increase in intensity or brightness (increased MPI) of the green dot fluorescence) over the control dogs (FIG. 6). However, there was no increase in the number or size of the fluorescent dots, indicating that peroxisome proliferation did not occur.

Analysis of Dog Hepatocyte Fluorescence by LSC:

There was a significant increase in AOX activity in the liver of ⅔ peroxisome proliferator-treated dogs (10.3-25.1% of hepatocytes with induced AOX activity compared to 0.3-4.6% in control females) (data not shown).

Equivalents

Those skilled in the art will recognize, or be able to ascertain using no more than routine experimentation, many equivalents of the specific embodiments of the invention described herein. Such equivalents are intended to be encompassed by the following claims.

1. A method for determining whether a test compound is capable of causing peroxisomal induction, comprising the steps of:
a) detectably labeling a first tissue sample obtained from a test organism and a second tissue sample obtained from a control organism with a peroxisome-specific antibody, said test organism having been treated with a test compound suspected of causing peroxisomal proliferation and induction;
b) measuring the intensity of the labeling of said first detectably-labeled tissue sample and the intensity of the labeling of said second detectably-labeled tissue sample using laser scanning cytometry; and
c) comparing the intensity of labeling of said first detectably-labeled tissue sample with the intensity of labeling of said second detectably-labeled tissue sample, wherein a greater intensity of labeling of said first detectably-labeled tissue sample relative to said second detectably-labeled tissue sample is indicative of the test compound causing peroxisomal induction.

2. A method for determining whether a test compound is capable of causing peroxisomal proliferation, comprising the steps of:
a) detectably labeling a first tissue sample obtained from a test organism and a second tissue sample obtained from a control organism with a peroxisome-specific antibody, said test organism having been treated with a test compound suspected of causing peroxisomal proliferation and induction;
b) determining the extent of the labeling of said first detectably-labeled tissue sample and the extent of the labeling of said second detectably-labeled tissue sample using laser scanning cytometry; and
c) comparing the number of labeled peroxisomes present in said first detectably-labeled tissue sample with the number of labeled peroxisomes present in said second detectably-labeled tissue sample, wherein a greater number of labeled peroxisomes present in said first detectably-labeled tissue sample relative to said second detectably-labeled tissue sample is indicative of the test compound causing peroxisomal proliferation.

3. A method for determining whether a test organism has been exposed to a compound capable of causing peroxisomal induction, comprising the steps of:
a) detectably labeling a first tissue sample obtained from a test organism and a second tissue sample obtained from a control organism with a peroxisome-specific antibody;
b) measuring the intensity of the labeling of said first detectably-labeled tissue sample and the intensity of the labeling of said second detectably-labeled tissue sample using laser scanning cytometry; and
c) comparing the intensity of labeling of said first detectably-labeled tissue sample with the intensity of labeling of said second detectably-labeled tissue sample, wherein a greater intensity of labeling of said first detectably-labeled tissue sample relative to said second detectably-labeled tissue sample is indicative of the test organism having been exposed to a compound capable of causing peroxisomal induction.

4. A method for determining whether a test organism has been exposed to a compound capable of causing peroxisomal proliferation, comprising the steps of:
a) detectably labeling a first tissue sample obtained from a test organism and a second tissue sample obtained from a control organism with a peroxisome-specific antibody;
b) determining the extent of the labeling of said first detectably-labeled tissue sample and the extent of the labeling of said second detectably-labeled tissue sample using laser scanning cytometry; and
c) comparing the number of labeled peroxisomes present in said first detectably-labeled tissue sample with the number of labeled peroxisomes present in said second detectably-labeled tissue sample, wherein a greater number of labeled peroxisomes present in said first detectably-labeled tissue sample relative to said second detectably-labeled tissue sample is indicative of the test organism having been exposed to a compound capable of causing peroxisomal proliferation.

5. The method according to any one of claims 1-4, wherein the test organism is an animal.

6. The method according to any one of claims 1-4, wherein the test organism is a rat.

7. The method according to any one of claims 1-4, wherein the test organism is a dog.

8. The method according to any one of claims 1-4, wherein the tissue sample is a liver tissue sample.

9. The method according to any one of claims 1-4, wherein the tissue sample is a heart tissue sample.

10. The method according to any one of claims 1-4, wherein the peroxisome-specific antibody is specific for the peroxisome targeting signal (PTS-1).

11. The method according to any one of claims 1-4, wherein the peroxisome-specific antibody is specific for a peroxisomal enzyme.

12. The method according to claim 11, wherein the peroxisomal enzyme is an oxidase.

13. The method according to claim 12, wherein the oxidase is acyl-CoA oxidase (AOX).

14. The method according to either claim 3 or claim 4, wherein the test organism is a human.

15. The method according to any one of claims 1-4, wherein the peroxisome-specific antibody is detectably labeled with a chromophore.

16. The method according to either claim 15, wherein the chromophore is fluorescein isothiocyanate (FITC).

17. The method according to either claim 16, wherein the chromophore is 4′,6-diamidino-2-phenylindole (DAP1).

18. A method of detecting whether peroxisomal induction has occurred in a test organism, comprising the steps of:
a) detectably labeling a tissue sample obtained from a test organism with a peroxisome-specific antibody;
b) measuring the intensity of the labeling of said detectably-labeled tissue using laser scanning cytometry; and
c) comparing the intensity of labeling of said detectably-labeled tissue sample with a known value for the intensity of labeling of a normal tissue sample, wherein a greater intensity of labeling of said detectably-labeled tissue sample relative to said known value for the intensity of labeling of a normal tissue sample is indicative of the test compound causing peroxisomal induction.

19. A method of detecting whether peroxisome proliferation has occurred in a test organism, comprising the steps of:
a) detectably labeling a tissue sample obtained from a test organism with a peroxisome-specific antibody;
b) determining the extent of the labeling of said detectably-labeled tissue sample using laser scanning cytometry; and
c) comparing the number of labeled peroxisomes present in said detectably-labeled tissue sample with a known number of labeled peroxisomes present in a normal tissue sample, wherein a greater number of labeled peroxisomes present in said detectably-labeled tissue sample relative to said known number of labeled peroxisomes present in a normal tissue sample is indicative of the test compound causing peroxisomal proliferation.
